(19) United States
(12) Patent Application Publication
(10) Pub. No.: US 2010/0120130 A1
(43) Pub. Date: May 13, 2010

(54) Droplet Actuator with Droplet Retention Structures

(75) Inventors: Vijay Srinivasan, Durham, NC (US); Vamsee K. Pamula, Durham, NC (US); Ramakrishna Sista, Morrisville, NC (US); Prasanna Thwar, Morrisville, NC (US)

Correspondence Address: ADVANCED LIQUID LOGIC, INC.;C/O WARD AND SMITH, P.A., 1001 COLLEGE COURT, P.O. BOX 867, NEW BERN, NC, 28563-0867, US

(73) Assignee: Advanced Liquid Logic, Inc., Research Triangle Park, NC (US)

(21) Appl. No.: 12/647,768

(22) Filed: Dec. 28, 2009

Related U.S. Application Data
(63) Continuation-in-part of application No. PCT/US2008/072604, filed on Aug. 8, 2008.
(60) Provisional application No. 61/141,083, filed on Dec. 29, 2008.
(60) Provisional application No. 60/980,620, filed on Oct. 17, 2007.
(60) Provisional application No. 60/954,587, filed on Aug. 8, 2007.

(51) Int. Cl. C12M 1/00; B01F 13/00; B01J 19/08; B01L 3/02; G01N 27/26

(52) U.S. Cl. 435/283.1; 204/600; 204/450; 422/61; 204/627; 206/223

(57) ABSTRACT

The present invention is directed to droplet actuators with droplet retention structures, and methods related thereto. In an exemplary embodiment, the invention provides a droplet actuator with one or more substrates arranged to form a droplet operations gap comprising gap-facing surfaces; droplet operations electrodes configured to conduct droplet operations in the droplet operations gap; at least one barrier included on at least one of the substrate surfaces and having dimensions selected to: permit droplet transport from atop a first droplet operations electrode to a second droplet operations electrode when the second droplet operations electrode is activated; and prevent movement of a droplet from atop a first droplet operations electrode when the first and second droplet operations electrodes are inactive.

### 1 RELATED APPLICATIONS

In addition to the patent applications cited herein, each of which is incorporated herein by reference, this patent application is related to and claims priority to U.S. Provisional Patent Application No. 61/141,083, filed on Dec. 29, 2008, entitled “Enhancing and/or Maintaining Oil Film Stability in a Droplet Actuator,” the entire disclosure of which is incorporated herein by reference. This patent application is also a continuation-in-part of International Patent Application No. PCT/US2008/072604, entitled “Use of Additives for Enhancing Droplet Operations,” filed on Aug. 8, 2008, pending, which claims priority to, is related to, and incorporates by reference U.S. Provisional Patent Application Nos. 60/954,587, entitled “Use of Additives for Enhancing Droplet Actuation,” filed on Aug. 8, 2007, and 60/980,620, entitled “Use of Additives for Enhancing Droplet Actuation,” filed on Oct. 17, 2007.

### 2 FIELD OF THE INVENTION

The present invention generally relates to the field of conducting droplet operations in a droplet actuator. In particular, the present invention is directed to modified droplet actuators, fluids and methods for enhancing and/or maintaining oil film stability in a droplet actuator.

### 3 BACKGROUND OF THE INVENTION

Droplet actuators are used to conduct a variety of droplet operations. A droplet actuator typically includes two substrates separated by a gap. The substrates include electrodes for conducting droplet operations. The gap between the substrates is typically filled with a filler fluid that is immiscible with the fluid that is to be subjected to droplet operations. Droplet operations are controlled by electrodes associated with one or both of the substrates. In some applications, the filler fluid is an oil film. The maintenance of the oil film between the droplet and the surface of the droplet actuator is essential for optimum operation of the droplet actuator. A stabilized oil film leads to less contamination, such as contamination due to adsorption and resorption. Therefore, there is a need for improved methods for enhancing and/or maintaining oil film stability in a droplet actuator.

### 4 BRIEF DESCRIPTION OF THE INVENTION

The invention provides a droplet actuator with one or more substrates arranged to form a droplet operations gap comprising gap-facing surfaces; droplet operations electrodes configured to conduct droplet operations in the droplet operations gap; at least one barrier included on at least one of the substrate surfaces and having dimensions selected to: permit droplet transport from atop a first droplet operations electrode to a second droplet operations electrode when the second droplet operations electrode is activated; and prevent movement of a droplet from atop a first droplet operations electrode when the first and second droplet operations electrodes are inactive. For example, the at least one barrier may include a physical barrier and/or a chemical barrier. In some embodiments, the at least one barrier comprises a projection from one or more of the gap-facing surfaces. In some embodiments, the at least one barrier comprises a hydrophobic surface. For example, the projection may be formed by embossing the gap-facing surface. The projection may be a ridge or other elevated region relative to the plane of the droplet actuator surface. In some cases, the projection comprises an elevated region surrounding an indentation which is aligned with the first droplet operations electrode. In some cases, one or more projections is provided at each edge of a droplet operations electrode. In some cases, the invention provides an array of such electrodes and their associated barriers. The invention also provides a method of retaining a droplet in place atop an electrode, the method comprising a droplet actuator according as described herein, providing a droplet in the droplet operations gap atop the second electrode, activating the first electrode and deactivating the second electrode to cause a droplet to flow onto the first electrode, and deactivating the first electrode and permitting the barrier to retain the droplet atop the first electrode.

The droplet actuators of the invention may include a droplet operations gap comprising a filler fluid at least partially filling the gap. The gap may be established, for example, by one or more substrates. For example, the substrates may include a top substrate and a bottom substrate, or a single substrate comprising a droplet operations gap formed therein. The substrates may include electrodes configured for conducting droplet operations in the droplet operations gap. The substrates may include one or more dielectric layers atop droplet operations electrodes. The substrates may include one or more hydrophobic layers, providing the interior surfaces of the droplet operations gap with a hydrophobic character. A filler fluid, such as an oil filler fluid may be provided in the droplet operations gap. The filler fluid may be provided in an amount sufficient to fill at least a portion of the gap and surround at least a portion of droplets in the gap. Filler fluid may form a layer between the droplets and the inner surface of the droplet operations gap.

The droplet actuator may include a surfactant in the oil filler fluid for stabilizing a film of oil filler fluid separating droplets therein from the hydrophobic layers. The droplet actuator may include at least one layer of surfactant between the oil filler fluid and the respective surfaces of the top substrate and bottom substrate. The surfactant may include an oleophilic surfactant. The oleophilic surfactant may, in some cases, include at least one of sugar esters, glycerin fatty acid esters and fatty acid monoglycerides. The hydrophobic layers may, in some embodiments, include a fluorinated hydrophobic coating and the surfactant may include at least one of a fluorinated oil, a fluorinated surfactant and an oleophilic oil. The at least one layer of surfactant may include a first layer and a second layer, the first layer may include a fluorinated surfactant, and the second layer may include at least one of an oleophilic oil and a fluorinated surfactant. The at least one layer of surfactant may include multiple layers.

The droplets may, in some embodiments, include an aqueous phase for conducting droplet operations with the droplet operations electrodes. The droplets may be partially surrounded by the oil filler fluid. The droplets may be substantially surrounded by the oil filler fluid. The hydrophobic layers may, in some embodiments, include at least one of a hydrocarbon, a silicone and an organic hydrophobic coating. The gap may have a height between the surfaces sufficient to maintain an oil film between droplets and the droplet operations electrodes.

The invention provides a droplet actuator for conducting droplet operations that includes a substrate having droplet operations electrodes on a surface thereof, a hydrophobic layer on at least one of the droplet operations electrodes, and an oil fluid on the surface of the substrate in an amount sufficient to surround droplets on the surface in an amount separating the droplets from the droplet operations electrodes.

The invention provides a droplet actuator for conducting droplet operations that has a top substrate having a surface thereof, a bottom substrate having droplet operations electrodes on a surface facing and spaced from the surface of the top substrate to form a gap therebetween, and at least one barrier included on at least one of the top substrate surface or bottom substrate surface at the edges of, or between, respective droplet operations electrodes. A hydrophobic layer may be provided on at least one of the droplet operations electrodes and on the surface of the top substrate. An oil filler fluid may be provided in the gap in an amount sufficient to fill at least a portion of the gap and surround at least a portion of droplets in the gap in a manner separating the droplets from the droplet operations electrodes, and the hydrophobic layers having an affinity for the oil filler fluid. Generally speaking, the may have a size which may, for example, be sufficient to permit droplet transport from one electrode to another when the droplet operations electrodes are activated, and to prevent movement of a droplet from a droplet operations electrode when the droplet operations electrodes may be inactive. The at least one barrier may, for example, be formed by embossing. The at least one barrier may, for example, be located only adjacent electrodes where it may, for example, be likely a droplet will reside for a relatively prolonged period of time, when compared to other electrodes of the droplet operations electrodes.

Any of the droplet actuator configurations described herein may include a feedback and control mechanism for monitoring voltage applied to the droplet operations electrodes, and for controlling the voltage applied to not exceed a minimum level sufficient to carry out selected droplet operations. The feedback and control mechanism may, for example, be adapted to detect capacitance from a voltage applied. The method of conducting a droplet operation may include applying a minimum voltage required to conduct a predetermined droplet operation for maintaining stability of the oil, the method facilitated by using the feedback mechanism for detecting when the droplet operation is complete or is sufficiently underway to ensure completion. The method may include modulating a voltage used to conduct a predetermined droplet operation for maintaining stability of the oil. The method may include limiting the duration of a voltage used to conduct a predetermined droplet operation for maintaining the stability of the oil. The method may include monitoring droplet operations and adjusting an electrowetting voltage used to conduct a predetermined droplet operation in response to the monitoring.

The invention provides a droplet actuator for conducting droplet operations that has droplet operations surfaces and electrodes configured for conducting droplet operations on the surfaces, and further includes a voltage application device for applying selected electrowetting voltages to selected electrodes in the path sufficient to transport droplets along the electrodes. The voltage application device may, for example, be configured for applying an electrowetting voltage of about 50 to about 500 volts, or about 75 to about 250 volts, or about 125 volts to about 175 volts. The voltage may, for example, be about 150 volts. The droplet actuator may include a reservoir configured for being activated to dispense droplets into the droplet actuator upon a predetermined electrowetting voltage being applied thereto. The reservoir may, for example, be disposed on the bottom substrate. The reservoir may, for example, be disposed on the top substrate. The reservoir may, for example, have a size which is approximately the same as a unit sized droplet operations electrode. The reservoir may, for example, have a size which is larger than a unit sized droplet operations electrode. The reservoir may, for example, have a size which is smaller than a unit sized droplet operations electrode.

The droplet actuator may include a separate substrate mounted on top of the top substrate or integral with the top substrate, and including a reservoir for holding a volume of fluid. The reservoir may, for example, be associated with a fluid path coupling the reservoir with the droplet operations gap in proximity to, or along, a path of droplet operations electrodes. The opening in the top substrate may, for example, be substantially aligned with a reservoir electrode on the droplet actuator, the reservoir electrode arranged in sufficient proximity to at least one droplet operations electrode in the path for conducting droplet operations on fluid introduced into the gap through the opening in the top substrate. The droplet actuator may be adapted for having an electrowetting voltage applied at the reservoir electrode sufficient to dispense droplets from the reservoir electrode. The voltage may, for example, be about 200 volts to about 250 volts. The voltage may, for example, be about 225 volts. In various cases, the voltage applied at a reservoir electrode during droplet dispensing is greater than the voltage applied at a droplet operations electrode to facilitate droplet operations, such as transport, splitting or merging. For example, the voltage applied at a reservoir electrode during droplet dispensing may be at least 10 volts greater, or at least 25 volts greater, or at least about 50 volts greater than the voltage applied at a droplet operations electrode to facilitate droplet operations, such as transport, splitting or merging.

The droplet actuator may include an oil filler fluid in the gap, multiple paths of droplet operations electrodes, and the voltage application device adapted for applying different voltages to electrodes performing different functions. The droplet actuator may include a reservoir from which droplets may be dispensed, a reservoir electrode associated therewith, and a feedback mechanism for monitoring droplet operations. The voltage application device may be adapted for applying a voltage sufficient to disrupt the oil film and cause cleaning droplets to be transported along the electrodes in a manner maximizing contact of the cleaning droplets with droplet actuator surfaces. The method may include transporting a sample droplet at a voltage having a level and duration which may, for example, be the lowest sufficient to conduct a predetermined droplet operation. The method may include conducting a cleaning operation by transporting cleaning droplets using a voltage sufficiently high to maximize contact between the cleaning droplets with droplet actuator surfaces.

The invention may include a kit including a droplet actuator, including a droplet operations gap configured for conducting droplet operations, and a sealed container of degassed filler fluid. The sealed container may include a solidified wax layer arranged to prevent exposure of the degassed filler fluid to the atmosphere. The degassed filler fluid may include degassed oil. The degassed filler fluid may include degassed silicone oil. The droplet actuator may be configured for conducting a reaction at a temperature exceeding room temperature. The droplet actuator may be configured for conducting a reaction at a temperature exceeding 40° C., or exceeding 50° C., or exceeding 60° C., or exceeding 70° C., or exceeding 80° C., or exceeding 90° C., or exceeding 40° C. The droplet actuator may be configured for conducting a reaction at a temperature which does not exceed about 150° C., or which does not exceed about 110° C., or which does not exceed about 100° C. For the sake of clarity, it is intended that any combination of the foregoing minimums and maximums may be combined to establish ranges which are also within the scope of the invention.

Similarly, the kit may include reagents selected for conducting a reaction at a temperature exceeding 40° C., or exceeding 50° C., or exceeding 60° C., or exceeding 70° C., or exceeding 80° C., or exceeding 90° C., or exceeding 40° C. The kit may include reagents selected for conducting a reaction at a temperature exceeding 40° C.

The kit may include reagents selected for conducting a reaction at a temperature which does not exceed about 150° C., or which does not exceed about 110° C., or which does not exceed about 100° C. For the sake of clarity, it is intended that any combination of the foregoing minimums and maximums may be combined to establish ranges which are also within the scope of the invention. The kit may include nucleic acid amplification reagents. The kit may include PCR reagents.

### 5 DEFINITIONS

As used herein, the following terms have the meanings indicated.

“Activate” with reference to one or more electrodes means effecting a change in the electrical state of the one or more electrodes which, in the presence of a droplet, results in a droplet operation.

“Bead,” with respect to beads on a droplet actuator, means any bead or particle that is capable of interacting with a droplet on or in proximity with a droplet actuator. Beads may be any of a wide variety of shapes, such as spherical, generally spherical, egg shaped, disc shaped, cubical and other three dimensional shapes. The bead may, for example, be capable of being transported in a droplet on a droplet actuator or otherwise configured with respect to a droplet actuator in a manner which permits a droplet on the droplet actuator to be brought into contact with the bead, on the droplet actuator and/or off the droplet actuator. Beads may be manufactured using a wide variety of materials, including for example, resins, and polymers. The beads may be any suitable size, including for example, microbeads, microparticles, nanobeads and nanoparticles. In some cases, beads are magnetically responsive; in other cases beads are not significantly magnetically responsive. For magnetically responsive beads, the magnetically responsive material may constitute substantially all of a bead or one component only of a bead. The remainder of the bead may include, among other things, polymeric material, coatings, and moieties which permit attachment of an assay reagent. Examples of suitable magnetically responsive beads include flow cytometry microbeads, polystyrene microparticles and nanoparticles, functionalized polystyrene microparticles and nanoparticles, coated polystyrene microparticles and nanoparticles, silica microbeads, fluorescent microspheres and nanospheres, functionalized fluorescent microspheres and nanospheres, coated fluorescent microspheres and nanospheres, color dyed microparticles and nanoparticles, magnetic microparticles and nanoparticles, superparamagnetic microparticles and nanoparticles (e.g., DYNABEADS® particles, available from Invitrogen Corp., Carlsbad, Calif.), fluorescent microparticles and nanoparticles, coated magnetic microparticles and nanoparticles, ferromagnetic microparticles and nanoparticles, coated ferromagnetic microparticles and nanoparticles, and those described in U.S. Patent Publication No. 20050260686, entitled, “Multiplex flow assays preferably with magnetic particles as solid phase,” published on Nov. 24, 2005, the entire disclosure of which is incorporated herein by reference for its teaching concerning magnetically responsive materials and beads. Beads may be pre-coupled with a biomolecule (ligand). The ligand may, for example, be an antibody, protein or antigen, DNA/RNA probe or any other molecule with an affinity for the desired target. Examples of droplet actuator techniques for immobilizing magnetically responsive beads and/or non-magnetically responsive beads and/or conducting droplet operations protocols using beads are described in U.S. patent application Ser. No. 11/639,566, entitled “Droplet-Based Particle Sorting,” filed on Dec. 15, 2006; U.S. Patent Application No. 61/039,183, entitled “Multiplexing Bead Detection in a Single Droplet,” filed on Mar. 25, 2008; U.S. Patent Application No. 61/047,789, entitled “Droplet Actuator Devices and Droplet Operations Using Beads,” filed on Apr. 25, 2008; U.S. Patent Application No. 61/086,183, entitled “Droplet Actuator Devices and Methods for Manipulating Beads,” filed on Aug. 5, 2008; International Patent Application No. PCT/US2008/053545, entitled “Droplet Actuator Devices and Methods Employing Magnetic Beads,” filed on Feb. 11, 2008; International Patent Application No. PCT/US2008/058018, entitled “Bead-based Multiplexed Analytical Methods and Instrumentation,” filed on Mar. 24, 2008; International Patent Application No. PCT/US2008/058047, “Bead Sorting on a Droplet Actuator,” filed on Mar. 23, 2008; and International Patent Application No. PCT/US2006/047486, entitled “Droplet-based Biochemistry,” filed on Dec. 11, 2006; the entire disclosures of which are incorporated herein by reference.

“Droplet” means a volume of liquid on a droplet actuator that is at least partially bounded by filler fluid. For example, a droplet may be completely surrounded by filler fluid or may be bounded by filler fluid and one or more surfaces of the droplet actuator. Droplets may, for example, be aqueous or non-aqueous or may be mixtures or emulsions including aqueous and non-aqueous components. Droplets may take a wide variety of shapes; nonlimiting examples include generally disc shaped, slug shaped, truncated sphere, ellipsoid, spherical, partially compressed sphere, hemispherical, ovoid, cylindrical, and various shapes formed during droplet operations, such as merging or splitting or formed as a result of contact of such shapes with one or more surfaces of a droplet actuator. For examples of droplet fluids that may be subjected to droplet operations using the approach of the invention, see International Patent Application No. PCT/US 06/47486, entitled, “Droplet-Based Biochemistry,” filed on Dec. 11, 2006. In various embodiments, a droplet may include a biological sample, such as whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, liquids containing single or multiple cells, liquids containing organelles, fluidized tissues, fluidized organisms, liquids containing multi-celled organisms, biological swabs and biological washes. Moreover, a droplet may include a reagent, such as water, deionized water, saline solutions, acidic solutions, basic solutions, detergent solutions and/or buffers. Other examples of droplet contents include reagents, such as a reagent for a biochemical protocol, such as a nucleic acid amplification protocol, an affinity-based assay protocol, an enzymatic assay protocol, a sequencing protocol, and/or a protocol for analyses of biological fluids.

“Droplet Actuator” means a device for manipulating droplets. For examples of droplet actuators, see U.S. Pat. No. 6,911,132, entitled “Apparatus for Manipulating Droplets by Electrowetting-Based Techniques,” issued on Jun. 28, 2005 to Pamula et al.; U.S. patent application Ser. No. 11/343,284, entitled “Apparatuses and Methods for Manipulating Droplets on a Printed Circuit Board,” filed on filed on Jan. 30, 2006; U.S. Pat. Nos. 6,773,566, entitled “Electrostatic Actuators for Microfluidics and Methods for Using Same,” issued on Aug. 10, 2004 and 6,565,727, entitled “Actuators for Microfluidics Without Moving Parts,” issued on Jan. 24, 2000, both to Shenderov et al.; Pollack et al., International Patent Application No. PCT/US2006/047486, entitled “Droplet-Based Biochemistry,” filed on Dec. 11, 2006; and Roux et al., U.S. Patent Pub. No. 20050179746, entitled “Device for Controlling the Displacement of a Drop Between two or Several Solid Substrates,” published on Aug. 18, 2005; the disclosures of which are incorporated herein by reference. Certain droplet actuators will include a substrate, droplet operations electrodes associated with the substrate, one or more dielectric and/or hydrophobic layers atop the substrate and/or electrodes forming a droplet operations surface, and optionally, a top substrate separated from the droplet operations surface by a gap. One or more reference electrodes may be provided on the top and/or bottom substrates and/or in the gap. In various embodiments, the manipulation of droplets by a droplet actuator may be electrode mediated, e.g., electrowetting mediated or dielectrophoresis mediated or Coulombic force mediated. Examples of other methods of controlling fluid flow that may be used in the droplet actuators of the invention include devices that induce hydrodynamic fluidic pressure, such as those that operate on the basis of mechanical principles (e.g. external syringe pumps, pneumatic membrane pumps, vibrating membrane pumps, vacuum devices, centrifugal forces, piezoelectric/ultrasonic pumps and acoustic forces); electrical or magnetic principles (e.g. electroosmotic flow, electrokinetic pumps, ferrofluidic plugs, electrohydrodynamic pumps, attraction or repulsion using magnetic forces and magnetohydrodynamic pumps); thermodynamic principles (e.g. gas bubble generation/phase-change-induced volume expansion); other kinds of surface-wetting principles (e.g. electrowetting, and optoelectrowetting, as well as chemically, thermally, structurally and radioactively induced surface-tension gradients); gravity; surface tension (e.g., capillary action); electrostatic forces (e.g., electroosmotic flow); centrifugal flow (substrate disposed on a compact disc and rotated); magnetic forces (e.g., oscillating ions causes flow); magnetohydrodynamic forces; and vacuum or pressure differential. In certain embodiments, combinations of two or more of the foregoing techniques may be employed in droplet actuators of the invention.

“Droplet operation” means any manipulation of a droplet on a droplet actuator. A droplet operation may, for example, include: loading a droplet into the droplet actuator; dispensing one or more droplets from a source droplet; splitting, separating or dividing a droplet into two or more droplets; transporting a droplet from one location to another in any direction; merging or combining two or more droplets into a single droplet; diluting a droplet; mixing a droplet; agitating a droplet; deforming a droplet; retaining a droplet in position; incubating a droplet; heating a droplet; vaporizing a droplet; cooling a droplet; disposing of a droplet; transporting a droplet out of a droplet actuator; other droplet operations described herein; and/or any combination of the foregoing. The terms “merge,” “merging,” “combine,” “combining” and the like are used to describe the creation of one droplet from two or more droplets. It should be understood that when such a term is used in reference to two or more droplets, any combination of droplet operations that are sufficient to result in the combination of the two or more droplets into one droplet may be used. For example, “merging droplet A with droplet B,” can be achieved by transporting droplet A into contact with a stationary droplet B, transporting droplet B into contact with a stationary droplet A, or transporting droplets A and B into contact with each other. The terms “splitting,” “separating” and “dividing” are not intended to imply any particular outcome with respect to volume of the resulting droplets (i.e., the volume of the resulting droplets can be the same or different) or number of resulting droplets (the number of resulting droplets may be 2, 3, 4, 5 or more). The term “mixing” refers to droplet operations which result in more homogenous distribution of one or more components within a droplet. Examples of “loading” droplet operations include microdialysis loading, pressure assisted loading, robotic loading, passive loading, and pipette loading. Droplet operations may be electrode-mediated. In some cases, droplet operations are further facilitated by the use of hydrophilic and/or hydrophobic regions on surfaces and/or by physical obstacles.

“Filler fluid” means a fluid associated with a droplet operations substrate of a droplet actuator, which fluid is sufficiently immiscible with a droplet phase to render the droplet phase subject to electrode-mediated droplet operations. The filler fluid may, for example, be a low-viscosity oil, such as silicone oil. Other examples of filler fluids are provided in International Patent Application No. PCT/US2006/047486, entitled, “Droplet-Based Biochemistry,” filed on Dec. 11, 2006; International Patent Application No. PCT/US2008/072604, entitled “Use of additives for enhancing droplet actuation,” filed on Aug. 8, 2008; and U.S. Patent Publication No. 20080283414, entitled “Electrowetting Devices,” filed on May 17, 2007; the entire disclosures of which are incorporated herein by reference. The filler fluid may fill the entire gap of the droplet actuator or may coat one or more surfaces of the droplet actuator. Filler fluid may be conductive or non-conductive. In some cases, particularly where the filler fluid will be heated, it is useful to oil with reduced concentration of dissolved gasses. Thus, the filler fluid may include a degassed oil, such as a degassed silicon oil. In certain embodiments, the filler fluid is provided pre-loaded in the droplet operations gap. In other embodiments, a kit may be provided in which the filler fluid is provided in a separate container and loaded into the droplet operations gap prior to execution of an assay. For example, well-degassed oil may be provided in sealed glass vials. In some embodiments, a melted wax such as tetracosane may be provided on top of oil to form a solid wax seal. The oil-filled glass vial may be closed by a cap, e.g., with gas tight PTFE/silicone/PTFE septum in a vacuum glove box. The degassed oil is therefore protected by a solid wax layer and a gas tight cap, which is suitable for long-term storage. In another embodiment, degassed oil may be provided in a gas-tight syringe with dispensing valve. Commercially available gas-tight syringes are usually made of glass and make use of a PTFE plunger. In another embodiment, a droplet operations gap may be filled with a degassed oil filler fluid, and wax plugs may be used to seal openings and thereby seal openings into the droplet operations gap. For example, a droplet operations gap may be filled with degassed oil, and then drop melted wax such as with high carbon number alkanes may be deposited in all openings, such as openings leading from the droplet operations gap into external top substrate reservoirs. The density of the melted wax may be selected to be less that the density of the filler fluid so that a barrier layer will be formed on top of the filler fluid following solidification of the melted wax. The degassed filler fluid may be stored in the droplet actuator gap until use. The solid wax may be re-melted by heaters or physically melted to reopen the sealed openings, e.g., to permit loading of reagents and/or sample.

“Immobilize” with respect to magnetically responsive beads, means that the beads are substantially restrained in position in a droplet or in filler fluid on a droplet actuator. For example, in one embodiment, immobilized beads are sufficiently restrained in position to permit execution of a splitting operation on a droplet, yielding one droplet with substantially all of the beads and one droplet substantially lacking in the beads.

“Magnetically responsive” means responsive to a magnetic field. “Magnetically responsive beads” include or are composed of magnetically responsive materials. Examples of magnetically responsive materials include paramagnetic materials, ferromagnetic materials, ferrimagnetic materials, and metamagnetic materials. Examples of suitable paramagnetic materials include iron, nickel, and cobalt, as well as metal oxides, such as Fe₃O₄, BaFe₁₂O₁₉, CoO, NiO, Mn₂O₃, Cr₂O₃, and CoMnP.

“Washing” with respect to washing a bead means reducing the amount and/or concentration of one or more substances in contact with the bead or exposed to the bead from a droplet in contact with the bead. The reduction in the amount and/or concentration of the substance may be partial, substantially complete, or even complete. The substance may be any of a wide variety of substances; examples include target substances for further analysis, and unwanted substances, such as components of a sample, contaminants, and/or excess reagent. In some embodiments, a washing operation begins with a starting droplet in contact with a magnetically responsive bead, where the droplet includes an initial amount and initial concentration of a substance. The washing operation may proceed using a variety of droplet operations. The washing operation may yield a droplet including the magnetically responsive bead, where the droplet has a total amount and/or concentration of the substance which is less than the initial amount and/or concentration of the substance. Examples of suitable washing techniques are described in Pamula et al., U.S. Pat. No. 7,439,014, entitled “Droplet-Based Surface Modification and Washing,” granted on Oct. 21, 2008, the entire disclosure of which is incorporated herein by reference.

The terms “top,” “bottom,” “over,” “under,” and “on” are used throughout the description with reference to the relative positions of components of the droplet actuator, such as relative positions of top and bottom substrates of the droplet actuator. It will be appreciated that the droplet actuator is functional regardless of its orientation in space.

When a liquid in any form (e.g., a droplet or a continuous body, whether moving or stationary) is described as being “on”, “at”, or “over” an electrode, array, matrix or surface, such liquid could be either in direct contact with the electrode/array/matrix/surface, or could be in contact with one or more layers or films that are interposed between the liquid and the electrode/array/matrix/surface.

When a droplet is described as being “on” or “loaded on” a droplet actuator, it should be understood that the droplet is arranged on the droplet actuator in a manner which facilitates using the droplet actuator to conduct one or more droplet operations on the droplet, the droplet is arranged on the droplet actuator in a manner which facilitates sensing of a property of or a signal from the droplet, and/or the droplet has been subjected to a droplet operation on the droplet actuator.

### 6 BRIEF DESCRIPTION OF THE DRAWINGS

FIG. 1 illustrates a side view of a portion of a droplet actuator, showing an oil film between the droplet and the surfaces of the droplet actuator;

FIG. 2 illustrates a side view of a portion of a droplet actuator that includes layered filler fluids for assisting to maintain the stability of the oil film;

FIG. 3 illustrates a side view of a portion of a droplet actuator that includes physical structures for droplet retention;

FIG. 4A illustrates a side view of a portion of a droplet actuator that includes a droplet transport region that requires a certain electrowetting voltage for transporting droplets;

FIG. 4B illustrates a side view of another portion of the droplet actuator of FIG. 4A that includes an on-droplet actuator reservoir that requires a certain electrowetting voltage for dispensing droplets;

FIG. 4C illustrates a side view of yet another portion of the droplet actuator of FIG. 4A that includes an off-droplet actuator reservoir that requires yet another certain electrowetting voltage for dispensing droplets; and

FIG. 5 illustrates a top view of the droplet actuator of FIGS. 4A, 4B, and 4C and shows the different regions therein that may require different voltages.

### 7 DETAILED DESCRIPTION OF THE INVENTION

The present invention provides modified droplet actuators, fluids and methods for enhancing and/or maintaining oil film stability in a droplet actuator. The maintenance of the oil film between the droplet and the surface of the droplet actuator is an important factor in optimum operation of the droplet actuator. A stabilized oil film leads to less contamination, such as contamination due to absorption and resorption. In addition, maintenance of the oil film provides for more direct electrowetting and allows for the use of lower voltages for droplet operations.

7.1 Droplet Actuator Structure with Surfactant Layers

Key parameters for maintaining the stability of the oil film in a droplet actuator include interfacial tension between the oil film (i.e., oil phase) and the surface of the droplet actuator (i.e., solid phase), and interfacial tension between liquid (i.e., aqueous phase) and the surface of the droplet actuator, the viscosity of the oil phase, the applied voltage, and the size of the gap between the top and bottom substrates of the droplet actuator.

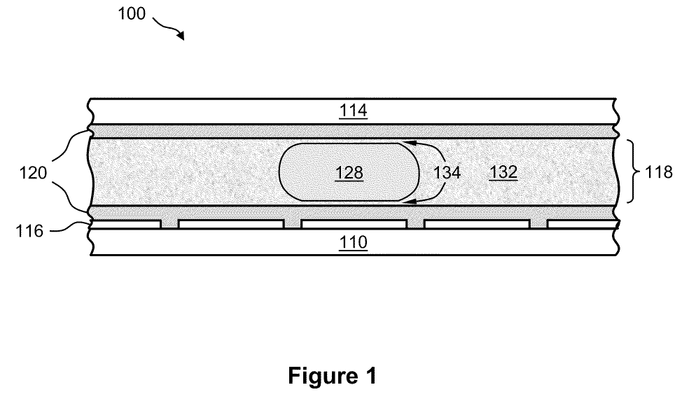

FIG. 1 illustrates a side view of a portion of a droplet actuator 100, showing an oil film between the droplet and the surfaces of the droplet actuator. Droplet actuator 100 may include a bottom substrate 110 that is separated from a top substrate 114 by a gap 118. A set of droplet operations electrodes 116, e.g., electrowetting electrodes, are arranged, for example, on bottom substrate 110. The droplet operations electrodes 116 are arranged for conducting droplet operations.

Bottom substrate 110 and top substrate 114 may include various coatings, typically including an outer hydrophobic coating, such as a fluoropolymer, such as a TEFLON® coating or a CYTOP® coating. A dielectric layer may be provided atop electrodes on bottom substrate 110. Top substrate 114 may include a ground, such as an ITO, conductive polymer or conductive ink, underlying a thin hydrophobic coating. A wide variety of materials are suitable for top substrate 114. The material selected must typically be smooth, permitting a fairly uniform gap height to be established. Examples of suitable materials include polymers, such as cyclic olephin copolymer and polycarbonate, and glass. For example, in one embodiment, the top substrate is comprised of 1.6 mm cyclic olephin copolymer, 1.6 mm polycarbonate, or 1.1 mm glass.

In some cases, the ground on top substrate 114 is electrically coupled to a ground on bottom substrate 110. Any suitable conductor may be used to establish this connection. In one embodiment, a conductive foam or a conductive elastomer is used. Nonlimiting examples of a suitable material is 500 μm thick EC-2040 from Rogers Corporation; silver paint, which may be air dried or thermally cured; and conductive epoxies.

The inventors have found that polycarbonate top-plates have a very low transmittance in the UV (below 400 nm the transmittance drastically reduces from 78% at 400 nm to 0.05% at 370 nm), rendering it unsuitable for UV fluorescence. Cyclic olephin copolymer at 1.6 mm has a significantly better transmission of 88% at 400 nm, reducing to 83% at 360 nm; however, COC has relatively high fluorescence in the UV with peak counts of ˜2000 diminishing to ˜1200 counts after considerable photobleaching—still too high for most umbelliferone assays which have background signal as low as 30 counts. Fluorinated polymers (“e.g.” TEFLON® AF, fluorinated ethylene propylene, CYTOP coatings) and silicones (PDMS) have much better optical transmission and autofluorescence specifications in the UV; however, due to reasons of manufacturing costs, injection molding a complete top substrate with a fluorinated polymer is not ideal. In certain embodiments, it is desirable to reduce or eliminate autofluorescence with minimal low-autofluorescence (fluorinated) polymer usage. In some cases, use of expensive polymers may be restricted to the detection regions only. Ideally, the polymers selected are low autofluorescence polymers as LFPs (Low Fluorescence Polymers). Low-cost injection molded polymers are ideal for such embodiments; examples include PC (polycarbonate), acrylic, cyclic olephin copolymer, or cyclic olephin copolymer.

In one embodiment, a polymeric top substrate may be provided with a glass surface. For example, a glass slide may be press-fit or adhered in a bottom side cutout of the top substrate. The glass slide may be retained in place by including the glass slide in the top substrate injection mold manufacture. This method is ideal from the optical standpoint, as glass has been shown in experimental studies to have low autofluorescence (and, of course, high optical transmission) in the 350-800 nm UV/VIS range. Likewise, a process established to incorporate glass in the top-substrate would work across all fluorophores and chemiluminescent substrates in the visible spectrum. In one embodiment, the invention provides for molding in place or adhering in a cutaway region of the same size/thickness, a glass microscope slide or coverslip (borosilicate glass) which covers the detection region. Since microscope slides are already well commercialized they can be obtained in large quantities at low cost. The challenge in this scenario is producing a surface flush (with a ˜25 um tolerance) with the bottom surface of the top-substrate. However, if the detection regime is at one end of droplet actuator, this flush requirement might only be required for one leading edge of the glass side, since any thickness variation in the detection zone can be normalized out by a calibration droplet.

In another embodiment, top substrate 114 may include openings into which droplets are transported for detection, such that during detection, no surface separates the droplet from the detector. A small ridge on the top substrate or a hydrophilic coating may be incorporated to allow for oil to fill into but not overflow the perimeter of the opening. A droplet which is lighter than the filler oil will “float” to the top of this opening. In some cases, the droplet will remain covered by a thin film of oil. Detection would occur directly at this opening.

Returning to the embodiment shown in FIG. 1, a hydrophobic layer 120 is disposed on the surface of bottom substrate 110 that is facing gap 118 (i.e., atop droplet operations electrodes 116). Similarly, another hydrophobic layer 120 is disposed on the surface of top substrate 114 that is facing gap 118. Hydrophobic layer 120 may be formed of, for example, a fluorinated hydrophobic coating, a hydrocarbon coating, a silicone coating, and/or an organic hydrophobic coating. Hydrophobic layer 120 has an affinity for an oil filler fluid 132 that is in gap 118. Hydrophobic layer 120 repels aqueous liquids, such as aqueous droplets that may be present along gap 118.

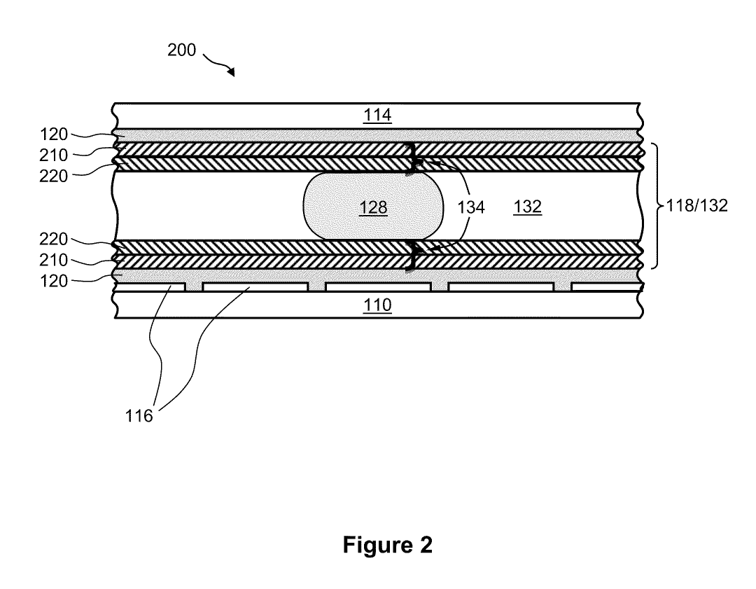

In one example, a droplet 128 may be present in gap 118 of droplet actuator 100. Droplet 128 may, for example, be a droplet of sample fluid or a reagent. Oil filler fluid 132 may, for example, be low-viscosity oil, such as silicone oil. Oil filler fluid 132 fills gap 118 and surrounds droplet 128. As droplet 128 moves along gap 118, an oil film 134 of oil filler fluid 132 forms between droplet 128 and the surfaces of droplet actuator 100. The stability of oil film 134 of oil filler fluid 132 that separates droplet 128 from hydrophobic layers 120 is important for optimum operation of droplet actuator 100. The stability of oil film 134 may be increased, for example, by decreasing the interfacial tension between oil filler fluid 132 and the surfaces within droplet actuator 100. In one embodiment, interfacial tension between oil filler fluid 132 (oil phase) and the surfaces within droplet actuator 100 (solid phase) may be modified by the addition of a surfactant to the oil filler fluid 132 within droplet actuator 100. An example of a droplet actuator that has additional filler fluid is described in more detail in FIG. 2.

FIG. 2 illustrates a side view of a portion of a droplet actuator 200 that includes filler fluid 132 including multilayered surfactants 210 and 220 for improving the stability of oil film 134. Droplet actuator 200 is substantially the same as droplet actuator 100 of FIG. 1, except that surfactant layers 210 and 220 are illustrated within filler fluid 132. Surfactant layers 210 and 220 may improve stability of the oil film 134. Thicknesses of the surfactant layers 210 and 220 are not to scale. Top substrate 114 and top surfactant layers 210 and 220 are illustrated, but are not required. Filler fluid 132 substantially fills gap 118, but complete filling of the gap with filler fluid 132 is not required.

In one embodiment, filler fluid 132 may include first surfactant layer 210 and a second surfactant layer 220. First surfactant layer 210 may be generally oriented atop hydrophobic layer 120. Second surfactant layer 220 may be generally oriented atop first surfactant layer 210. It will be appreciated that in addition to the layers illustrated, some portion of one or both surfactants may be distributed elsewhere in filler fluid 132. Droplet 128 provides an aqueous phase for conducting droplet operations mediated by electrodes 116. Droplet 128 may be partially surrounded by filler fluid 132. Alternatively, droplet 128 may be substantially surrounded by filler fluid 132.

In one embodiment, hydrophobic layer 120 includes a fluorinated hydrophobic coating. In a related embodiment, hydrophobic layer 120 includes a fluorinated hydrophobic coating and first surfactant 210 includes a fluorinated oil. In another related embodiment, hydrophobic layer 120 includes a fluorinated hydrophobic coating, first surfactant 210 includes a fluorinated surfactant, and second surfactant 220 includes an oleophilic oil.

In another embodiment, hydrophobic layer 120 includes a hydrocarbon, a silicone, and/or an organic hydrophobic coating. In a related embodiment, hydrophobic layer 120 includes a hydrocarbon, a silicone, and/or an organic hydrophobic coating and first surfactant 210 includes fluorinated surfactant. In another related embodiment, hydrophobic layer 120 includes a hydrocarbon, a silicone, and/or an organic hydrophobic coating, first surfactant 210 includes a fluorinated surfactant, and second surfactant 210 includes a fluorinated surfactant.

Examples of suitable oleophilic surfactants include, without limitation, sugar esters, such as sorbitan fatty acid esters (e.g., sorbitantrioleate, sorbitantrilaurate, sorbitantripalmitate, sorbitantristearate and sorbitantrisesquioleate) and sucrose fatty acid esters; glycerin fatty acid esters; and fatty acid monoglycerides.

Examples of suitable fluorinated surfactants include, without limitation, 1H,1H,2H,2H-perfluoro-1-decanol and 1H,1H,2H,2H-perfluoro-1-octanol; as well as perfluorinated surfactants, such as perfluorodecanoic acid and perfluorododecanoic acid. A list of fluorinated surfactants is available in Chapter 1 “Fluorinated Surfactants and Repellents” By Erik Kissa, Published by CRC Press, 2001, the entire disclosure of which is incorporated herein by reference. Other suitable fluorinated surfactants are described in Michael Terrazas & Rudi Dams, “A new generation of fluorosurfactants,” Specialty Chemicals Magazine, March 2004, vol. 24, no. 3, the entire disclosure of which is incorporated herein by reference.

### 7.2 Filler Fluid Viscosity

The stability of the oil film may be increased by increasing the interfacial tension between droplet 128 (the aqueous phase) and hydrophobic layer 120 (the solid phase). In one embodiment, the invention comprises selecting an oil filler fluid having sufficiently high viscosity to maintain the integrity of the oil film during the conduct of one or more droplet operations.

### 7.3 Gap Height

Increasing the size of gap 118, i.e., the distance between bottom substrate 110 and top substrate 114, results in a decrease in the interfacial tension between the oil phase and solid phase, which increases the stability of the oil film. The invention may comprise selecting a gap height which is sufficiently large relative to the unit droplet size to maintain the integrity of the oil film during the conduct of one or more droplet operations. The unit droplet has a footprint which is roughly the same as the footprint of a unit sized droplet operations electrode. In one embodiment, top substrate 114 may be omitted altogether.

7.4 Droplet Actuator Structure with Barriers

Lengthy electrode activation may be detrimental to oil film stability. Consequently, it may be useful in some cases to minimize the length of time that an electrode is activated. Current techniques activate an electrode to move a droplet into place atop the electrode and to retain the droplet in place. The invention includes a technique whereby electrode activation is used to move a droplet into place, while physical barriers are used to retain the droplet in place. In this manner, the duration of electrode activation may be limited to the duration necessary to move the droplet into place.

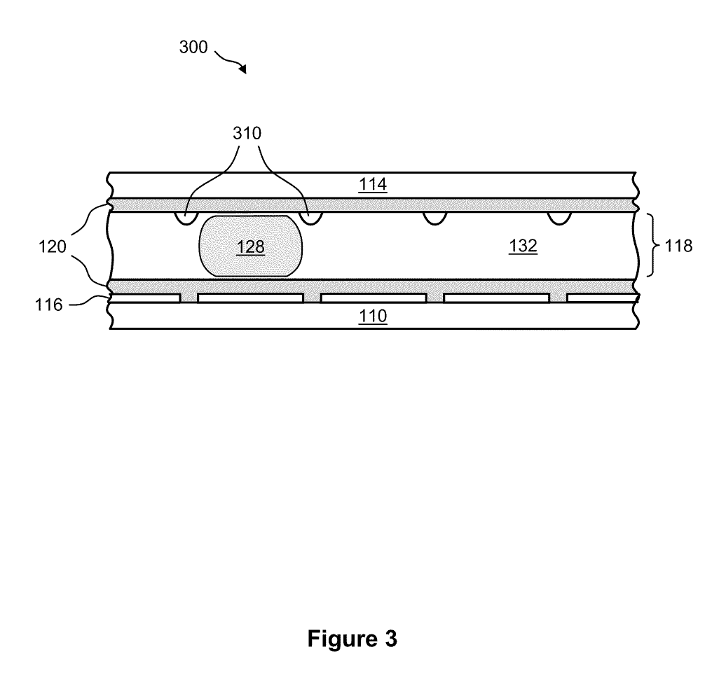

FIG. 3 illustrates a side view of a portion of a droplet actuator 300 that includes physical structures for droplet retention. Droplet actuator 300 may be substantially the same as droplet actuator 100 of FIG. 1, except for the inclusion of barriers 310 on, for example, the surface of top substrate 114 that is facing gap 118. Barriers 310 may be physical structures that are placed approximately at the edges of or between droplet operations electrodes 116. Barriers 310 may be formed by, for example, embossing. Barriers 310 are designed to permit droplet transport, while at the same time hinder droplet drift in the absence of an activated electrode. Other types of physical features may be used so long as they permit droplet transport, while at the same time hinder droplet drift in the absence of an activated electrode. For example, the physical barriers may be replaced with a divot in the top and/or bottom substrates.

In operation, droplet operations electrodes 116 of droplet actuator 300 may be activated to transport droplet 128. Subsequent to transport of droplet 128, droplet operations electrode 116 may be deactivated. Droplet 128 is then prevented by barriers 310 from drifting away from droplet operations electrode 116. Barriers 310 are provided in order to retain droplet 128 on a certain droplet operations electrode 116 even in the absence of an applied electrowetting voltage. As a result, the presence of barriers 310 allows the applied electrowetting voltage to be removed and/or reduced upon completion of the droplet operations, thereby helping to maintain the stability of the oil film.

In one embodiment, structures for maintaining a droplet in place are included at locations in the droplet actuator where it is likely that a droplet will need to reside for a prolonged period of time, i.e., more than 0.1 seconds or more than 0.5 seconds or more than 1 second. For example, structures for maintaining a droplet in place may be provided within a detection window so that electrowetting voltage is not required to maintain a droplet in place during a detection operation. In this manner, contamination in a detection window may be reduced relative to contamination that would occur if the droplet were maintained in place by electrowetting during the detection operation.

### 7.5 Adjustable Electrowetting Voltages in a Droplet Actuator

Modulating the voltage used to perform droplet operations may assist in maintaining the stability of the oil film. In general, minimizing the voltage level of the electrowetting voltage and/or the duration that the voltage is applied during droplet operations may be beneficial for maintaining the stability of the oil film.

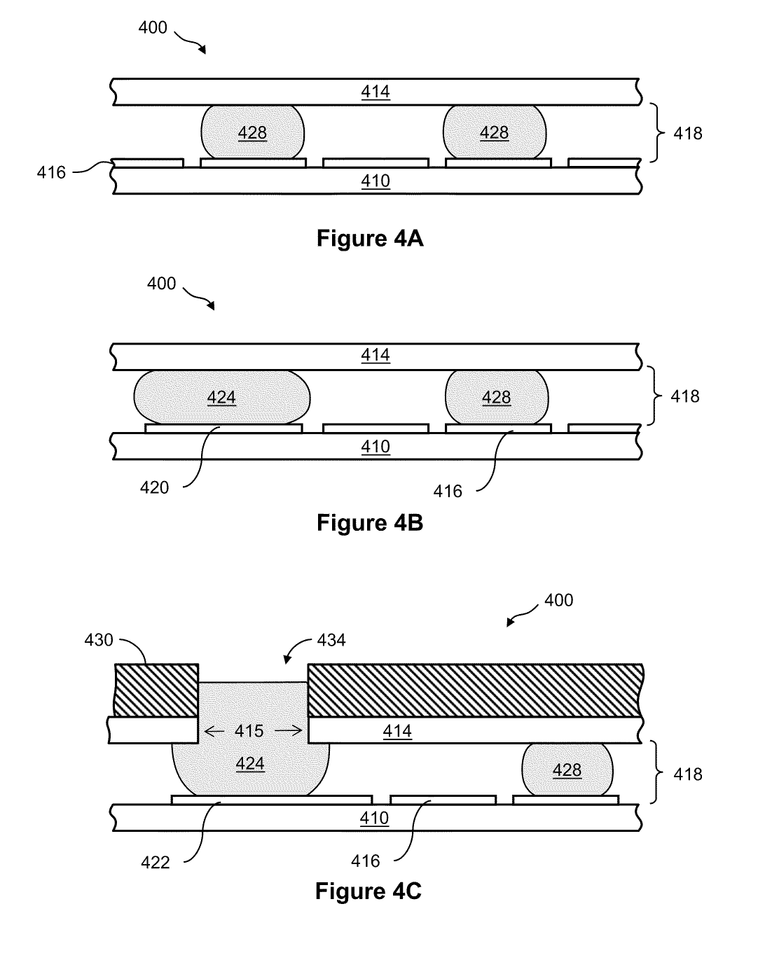

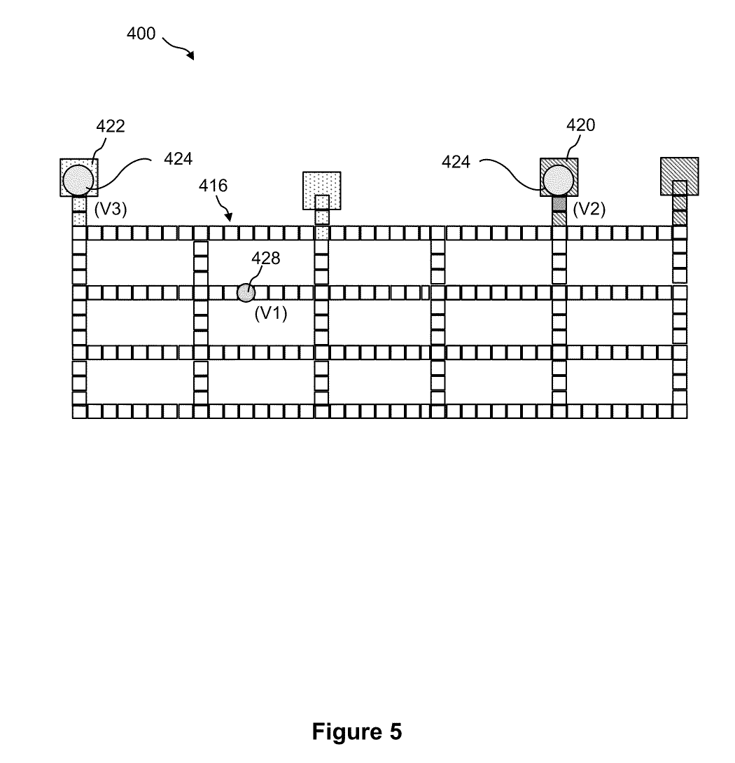

Embodiments of the invention may utilize certain feedback mechanisms for monitoring droplet operations and adjusting the electrowetting voltage accordingly. Using substantially continuous feedback mechanisms permits voltage duration to be reduced to the duration necessary to carry out a certain droplet operation. In one example, capacitance detection may be used as the substantially continuous feedback mechanism. Examples of capacitance feedback mechanisms suitable for use in the present invention are described in International Patent Application No. PCT/US08/54134, entitled “Capacitance Detection in a Droplet Microactuator,” filed on Feb. 15, 2008, the entire disclosure of which is incorporated herein by reference. In another example, an optical feedback system, such as a camera in combination with image processing technologies, may be used as the substantially continuous feedback mechanism. Examples of using adjustable electrowetting voltages to help maintain the stability of the oil film are described with reference to FIGS. 4A, 4B, 4C, and 5.

FIG. 4A illustrates a side view of a section of a droplet actuator 400. This section of droplet actuator 400 includes a droplet transport region that requires a certain electrowetting voltage for transporting droplets. Droplet actuator 400 may include a bottom substrate 410. Bottom substrate 410 may be separated from a top substrate 414 by a gap 418. The transport region of droplet actuator 400 may include a line or path of droplet operations electrodes 416 (e.g., electrowetting electrodes) that may be associated with bottom substrate 410. One or more droplets 428 may be contained in gap 418 of droplet actuator 400. In order to transport droplets 428 along droplet operations electrodes 416, a certain electrowetting voltage is applied. For example, an electrowetting voltage V1 from about 125 volts to about 175 volts (e.g., about 150 volts) may be sufficient for transporting droplets along droplet operations electrodes 416.

FIG. 4B illustrates a side view of another portion of droplet actuator 400. This portion of droplet actuator 400 includes an on-droplet actuator reservoir that requires a certain electrowetting voltage for dispensing droplets. An on-droplet actuator reservoir electrode 420 may be disposed on bottom substrate 410. On-droplet actuator reservoir electrode 420 may be arranged in association with the line or path of droplet operations electrodes 416. On-droplet actuator reservoir electrode 420 is illustrated as being larger than droplet operations electrodes 416, but may be the same size or smaller. In some cases, on-droplet actuator reservoir electrode 420 is simply replaced with another droplet operations electrode 416.

Droplets may be dispensed from on-droplet actuator reservoir electrode 420 onto the droplet operations electrodes 416. More specifically, a volume of sample fluid 424 is provided at on-droplet actuator reservoir electrode 420. Droplets, such as a droplet 428, may be dispensed from sample fluid 424 by applying a certain electrowetting voltage. For example, an electrowetting voltage V2 from about 150 volts to about 200 volts (e.g., about 175 volts) may be sufficient for dispensing droplets from on-droplet actuator reservoir electrode 420.

FIG. 4C illustrates a side view of yet another portion of droplet actuator 400. This portion of droplet actuator 400 includes an off-droplet actuator reservoir that requires yet another electrowetting voltage for dispensing droplets. A substrate 430, such a plastic substrate, is mounted atop top substrate 414. Substrate 430 includes a reservoir 434 for holding a volume of fluid 424. Reservoir 434 is substantially aligned with an opening 415 in top substrate 414. Additionally, the opening in top substrate 414 is substantially aligned with an reservoir electrode 422, which may be disposed on bottom substrate 410. Reservoir electrode 422 may be arranged in sufficient proximity to one or more electrodes in the line or path of droplet operations electrodes 416 such that the one or more electrodes may be used to conduct one or more droplet operations using fluid 424 introduced into gap 418 via opening 415. Reservoir electrode 422 is illustrated as being larger than droplet operations electrodes 416, but may be the same size or smaller. In some cases, reservoir electrode 422 is simply replaced with another droplet operations electrode 416. The fluid path from reservoir 434 into gap 418 permits reservoir electrode 422 to interact with fluid 424. Fluid 424 may, for example, be a wash fluid or a sample fluid.

In this example, wash droplets may be dispensed from reservoir electrode 422 onto the droplet operations electrode 416. More specifically, a volume of fluid 424 is provided at reservoir electrode 422. Droplets 428, which may be wash droplets, may be dispensed from fluid 424 by applying a certain electrowetting voltage. For example, an electrowetting voltage V3 from about 200 volts to about 250 volts (e.g., about 225 volts) may be sufficient for dispensing droplets from reservoir electrode 422.

Referring to FIGS. 4A, 4B, and 4C, a higher voltage may be required to pull fluid into the gap and to subsequently dispense droplets from an reservoir electrode (e.g., V3 of FIG. 4C) as compared with an on-droplet actuator reservoir electrode (e.g., V2 of FIG. 4B), and as compared to the droplet transport operations (e.g., V1 of FIG. 4A). In another example, even lower voltages (e.g., V0) than the voltage V1 that is sufficient for droplet transport may be required to prevent droplet drift (i.e., keeping a droplet in place). Voltage requirements for the different droplet operations of droplet actuator 400 may be described as V0≦V1≦V2≦V3. FIGS. 4A, 4B, and 4C describe examples wherein different voltage levels may be just sufficient (and with just sufficient time) to perform the certain droplet operations, which may be beneficial for maintaining the stability of the oil film. In one embodiment, the invention provides a droplet actuator configured for applying a voltage to each electrode, wherein the voltage applied to each electrode is selected to be the minimal voltage for the specific task being conducted by the electrode. In one such embodiment, the voltages applied are V0≦V1≦V2≦V3, as described above.

FIG. 5 illustrates a top view of droplet actuator 400 that is described in FIGS. 4A, 4B, and 4C and illustrates regions that may require different voltages. For example, FIG. 5 shows multiple lines or paths of droplet operations electrodes 416 along which droplets, such as droplet 428, may be transported using, for example, electrowetting voltage V1. Additionally, on-droplet actuator reservoir electrode 420 is shown, from which droplets may be dispensed using, for example, electrowetting voltage V2. Further, reservoir electrode 422 is shown, from which droplets may be dispensed using, for example, electrowetting voltage V3. Feedback mechanisms (not shown), such as capacitance detection and optical detection mechanisms, may be associated with droplet actuator 400 for monitoring droplet operations. By monitoring the droplet operations in a substantially continuous manner, the electrowetting voltage levels, the amount of time for applying the voltage levels, the voltage shape (i.e., waveform), location at which to apply the voltage, and so on, may be determined and controlled. For example, the minimum voltage and duration may be applied to perform a transport operation. Then, once it has been determined that the transport operation is complete, the voltage may be reduced or removed.

In one embodiment, different voltages may be applied to electrodes performing different functions. For example, when transporting a sample droplet, smaller or minimum voltages and voltage durations may be used to reduce contamination of the droplet actuator surface. Subsequent cleaning droplets may be transported using higher voltages in order to maximize contact of the cleaning droplet with the droplet actuator surface. In other words, in some cases, disrupting the oil film may be useful, particularly for clean-up purposes. It may also be useful to disrupt the oil film for depositing substances on a surface of the droplet actuator. The oil film may be disrupted by increasing voltage and/or voltage time. Further, the sample droplet may be followed by a low interfacial tension cleaning droplet so that whatever rupture in the oil film that the sample droplet may have caused is restored by the cleaning droplet, which picks up the contamination. In this example, the cleaning droplet has about the same characteristics as the sample droplet and, therefore, uses about the same voltage. Droplets with beads may be subjected to droplet operations using different voltages than corresponding droplets lacking beads. Voltages may vary with the type of droplet operation being performed.

### 7.6 Loading Oil

Bubbles usually form during oil loading when the oil front moves at different speeds on different sides of the droplet actuator and finally when the multiple fronts of oil come together, essentially engulfing a bubble in the process. One way to ensure oil flows uniformly in all directions is to load it from the middle of the droplet actuator so that it flows radially through the droplet operations gap. When oil is loaded from a side of a rectangle shaped droplet actuator from a position which is close to the gasket area, the oil can wick through the cracks in the gasket area and thus travel faster in one direction compared to flowing in the free area of the sandwich. Eventually this may lead to formation of a bubble. Reduction of bubble formation during loading may be achieved by placing the loading opening away from the gasket or any structures that can cause significant wicking, and placing the opening in the center (i.e., generally centrally located relative to the edges of the droplet operations gap that is being filled) is a further improvement.

In some cases, oil may be stored on in the droplet operations gap. In other cases, oil may be stored in an external reservoir and flowed into the droplet operations gap prior to execution of an assay protocol. In one embodiment, a reservoir in the top substrate may be provided with an opening for flowing oil into the droplet operations gap. This opening may be sealed by a removable or rupturable barrier, for example, by a rupturable film, such as a thin plastic laminated film. The top portion of this reservoir may be sealed with such a film after oil has been loaded. The droplet actuator may be transportable with oil sealed in the reservoir. At the user site, the user may activate the loading of oil by eliminating, removing or otherwise breaching the removable or rupturable barrier, thus releasing the oil to flow into the droplet operations gap.

### 8 CONCLUDING REMARKS

The foregoing detailed description of embodiments refers to the accompanying drawings, which illustrate specific embodiments of the invention. Other embodiments having different structures and operations do not depart from the scope of the present invention. The term “the invention” or the like is used with reference to certain specific examples of the many alternative aspects or embodiments of the applicants' invention set forth in this specification, and neither its use nor its absence is intended to limit the scope of the applicants' invention or the scope of the claims. This specification is divided into sections for the convenience of the reader only. Headings should not be construed as limiting of the scope of the invention. The definitions are intended as a part of the description of the invention. It will be understood that various details of the present invention may be changed without departing from the scope of the present invention. Furthermore, the foregoing description is for the purpose of illustration only, and not for the purpose of limitation.

We claim:

1. A droplet actuator comprising:
(a) one or more substrates arranged to form a droplet operations gap comprising gap-facing surfaces;
(b) droplet operations electrodes configured to conduct droplet operations in the droplet operations gap; and
(c) at least one barrier included on at least one of the substrate surfaces and having dimensions selected to:
(i) permit droplet transport from atop a first droplet operations electrode to a second droplet operations electrode when the second droplet operations electrode is activated; and
(ii) prevent movement of a droplet from atop a first droplet operations electrode when the first and second droplet operations electrodes are inactive.

2. The droplet actuator of claim 1 wherein the at least one barrier comprises a physical barrier.

3. The droplet actuator of claim 1 wherein the at least one barrier comprises a chemical barrier.

4. The droplet actuator of claim 1 wherein the at least one barrier comprises a projection from one or more of the gap-facing surfaces.

5. The droplet actuator of claim 1 wherein the at least one barrier comprises a hydrophobic surface.

6. The droplet actuator of claim 4 wherein the projection is formed by embossing the gap-facing surface.

7. The droplet actuator of claim 4 wherein the projection comprises a ridge.

8. The droplet actuator of claim 4 wherein the projection comprises an elevated region surrounding an indentation which is aligned with the first droplet operations electrode.

9. The droplet actuator of claim 1 comprising one or more projections at each edge of a droplet operations electrode.

10. The droplet actuator of claim 1 comprising an array of said first electrodes.

11. The droplet actuator of claim 1, further comprising a feedback and control mechanism for monitoring voltage applied to the droplet operations electrodes, and for controlling the voltage applied to not exceed a minimum level sufficient to carry out selected droplet operations.

12. The droplet actuator of claim 11, wherein the feedback and control mechanism is adapted to detect capacitance from a voltage applied.

13. A method of conducting droplet operations using the droplet actuator of claim 1, the method comprising applying a minimum voltage required to conduct a predetermined droplet operation for maintaining stability of the oil.

14. A method of conducting droplet operations using the droplet actuator of claim 1, the method comprising modulating a voltage used to conduct a predetermined droplet operation for maintaining stability of the oil.

15. A method of conducting droplet operations with the droplet actuator of claim 1, the method comprising limiting the duration of a voltage used to conduct a predetermined droplet operation for maintaining the stability of the oil.

16. A method of conducing droplet operations with the droplet actuator of claim 1, the method comprising monitoring droplet operations and adjusting an electrowetting voltage used to conduct a predetermined droplet operation in response to the monitoring.

17. A method of retaining a droplet in place atop an electrode, the method comprising:
(a) providing a droplet actuator according to claim 1;
(b) providing a droplet in the droplet operations gap atop the second electrode;
(c) activating the first electrode and deactivating the second electrode to cause a droplet to flow onto the first electrode;
(d) deactivating the first electrode and permitting the barrier to retain the droplet atop the first electrode.

18. A kit comprising:
(a) a droplet actuator comprising a droplet operations gap configured for conducting droplet operations;
(b) a sealed container of degassed filler fluid.

19. The kit of claim 18 wherein the sealed container comprises a solidified wax layer arranged to prevent exposure of the degassed filler fluid to the atmosphere.

20. The kit of claim 18 wherein the degassed filler fluid comprises degassed oil.

21. The kit of claim 18 wherein the degassed filler fluid comprises degassed silicone oil.

22. The kit of claim 18 wherein the droplet actuator is configured for conducting a reaction at a temperature exceeding room temperature.

23. The kit of claim 18 wherein the droplet actuator is configured for conducting a reaction at a temperature exceeding 40° C.

24. The kit of claim 18 wherein the droplet actuator is configured for conducting a reaction at a temperature exceeding 50° C.

25. The kit of claim 18 wherein the droplet actuator is configured for conducting a reaction at a temperature exceeding 60° C.

26. The kit of claim 18 wherein the droplet actuator is configured for conducting a reaction at a temperature exceeding 70° C.

27. The kit of claim 18 wherein the droplet actuator is configured for conducting a reaction at a temperature exceeding 80° C.

28. The kit of claim 18 wherein the droplet actuator is configured for conducting a reaction at a temperature exceeding 90° C.

29. The kit of claim 18 wherein the droplet actuator is configured for conducting a reaction at a temperature exceeding 40° C.

30. The kit of claim 18 wherein the droplet actuator is configured for conducting a reaction at a temperature which does not exceed about 150° C.

31. The kit of claim 18 wherein the droplet actuator is configured for conducting a reaction at a temperature which does not exceed about 110° C.

32. The kit of claim 18 wherein the droplet actuator is configured for conducting a reaction at a temperature which does not exceed about 100° C.

33. The kit of claim 18 wherein the kit comprises reagents selected for conducting a reaction at a temperature exceeding 40° C.

34. The kit of claim 18 wherein the kit comprises reagents selected for conducting a reaction at a temperature exceeding 50° C.

35. The kit of claim 18 wherein the kit comprises reagents selected for conducting a reaction at a temperature exceeding 60° C.

36. The kit of claim 18 wherein the kit comprises reagents selected for conducting a reaction at a temperature exceeding 70° C.

37. The kit of claim 18 wherein the kit comprises reagents selected for conducting a reaction at a temperature exceeding 80° C.

38. The kit of claim 18 wherein the kit comprises reagents selected for conducting a reaction at a temperature exceeding 90° C.

39. The kit of claim 18 wherein the kit comprises reagents selected for conducting a reaction at a temperature exceeding 40° C.

40. The kit of claim 18 wherein the kit comprises reagents selected for conducting a reaction at a temperature which does not exceed about 150° C.

41. The kit of claim 18 wherein the kit comprises reagents selected for conducting a reaction at a temperature which does not exceed about 110° C.

42. The kit of claim 18 wherein the kit comprises reagents selected for conducting a reaction at a temperature which does not exceed about 100° C.

43. The kit of claim 18 wherein the kit comprises nucleic acid amplification reagents.

44. The kit of claim 18 wherein the kit comprises PCR reagents.
